(19) United States
(12) Patent Application Publication
(10) Pub. No.: US 2006/0211969 A1
(43) Pub. Date: Sep. 21, 2006

(54) Patient support frame

(76) Inventors: Ross W. Terman, Rochester, MN (US); James K. Edvenson, Oronoco, MN (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(21) Appl. No.: 11/372,791

(22) Filed: Mar. 10, 2006

Related U.S. Application Data
(60) Provisional application No. 60/661,583, filed on Mar. 14, 2005.

(51) Int. Cl. A61F 5/00

(52) U.S. Cl. 602/32

(57) ABSTRACT

An appliance provides a pair of sidebars and a pair of cross-bars which are assembled around a patient reclining upon a supporting surface. The appliance has legs which support the appliance at the level of the patient. The cross-bars are positioned under the hips and shoulders of the patient. A flexible strap, which has tension adjustment apparatus, extends between the cross-bars under the patient's spine to assist in supporting the patient. After the appliance is assembled the supporting surface can be removed to provide complete access around the periphery of the patient for examination and treatment.

### CROSS-REFERENCE TO RELATED APPLICATIONS

This is a regular application filed under 35 U.S.C. § 111(a) claiming priority, under 35 U.S.C. § 119(e)(1), of provisional application Ser. No. 60/661,583, previously filed Mar. 14, 2005 under 35 U.S.C. § 111(b).

### TECHNICAL FIELD

The present invention deals with the field of medical furniture appliances. More specifically, however, it deals with frames used to support a patient in a position elevated with respect to a bed, gurney or table which normally receives the patient.

### BACKGROUND OF THE INVENTION

For various purposes, patients are positioned on a hospital bed, a gurney or a table such as an examination table or an operating table. It can be necessary to have substantially full 360° circumferential access about the patient. Systems heretofore require transferring of the patient to another bed or movement which can aggravate a medical condition or cause severe pain or discomfort. The present invention addresses these shortcomings of the prior art.

### SUMMARY OF THE INVENTION

The present invention is an appliance used to safely support a person while in a hospital bed, on a gurney or on a table. When the appliance is in position supporting the patient, the bed, gurney or table can be lowered from under the person. Substantially full circumferential access is thereby afforded to the person's torso for examination and/or treatment.

The appliance comprises a frame which includes a pair of longitudinally extending sidebars, one on each side of the patient. The longitudinally extending sidebars are, when the appliance is fully assembled, supported by appropriate means, such as a pair of generally vertically disposed legs, at a desired height, which can be adjusted for a variety of supports. Typically, the legs would intersect a corresponding sidebar at the ends and form a generally longitudinal inverted U-shape. The legs can be separable from the sidebars so that the frame, when not in use, can be fully disassembled, or can be integral with each other. Cross-bars are provided which extend perpendicularly between the sidebars. The sidebars are provided to support a patient shoulder and hip supporting cross-bars, which extend perpendicularly between the cross-bars. Multiple fittings can be provided along the sidebars to allow for adjustment of the separation of the shoulder bar from the hip bar for different patient heights. In one embodiment, the shoulder bar and the hip bar can each be formed from a spaced pair of ⅞ inch outside diameter tubing segments welded to each other in a parallel configuration utilizing a metal webbing. A threaded shaft can be arranged to engage a nut attached between the tubing ends to secure the bar in place.

In another embodiment the ends of the shoulder and hip bars can be attached to the sidebars by means of generally trapezoidally-shaped recesses formed in fittings carried by inwardly-facing surfaces of the sidebars. The smaller edge of the trapezoidal-shaped fitting is positioned at the bottom of the fitting, and this allows for a correspondingly-shaped trapezoidal member to be inserted downwardly and wedged into the fitting. It will be understood that, in this embodiment, each fitting would be provided with a lip which would securely prevent the shoulder bar and hip bar from sliding laterally out of a fitting carried by a longitudinally-extending sidebar.

Means can also be provided, in this embodiment, to preclude ends of the shoulder bar and hip bar from jumping up and out of the corresponding fittings in which they are received. Such means could take the form of an elastic bungee run over the top of the end of a shoulder bar or hip bar with ends of the bungee being captured at appropriate locations along a generally vertically-extending, proximate leg.

A central longitudinal spinal strap of flexible material is provided between the sidebars. The strap is in the form of an endless belt which has one end slid over the shoulder bar and lies on top of the hip bar. The other end of the belt has a tension bar slid through a loop in the end of the belt. Opposite ends of the tension bar each mount a longitudinally-extending threaded shaft. These threaded shafts extend in a generally longitudinal parallel fashion and have a nut arranged to provide means to adjust the strap tension and to preclude shortening of the distance of the tension bar from the hip bar. The end of the spinal strap opposite the shoulder bar has the tension bar attached. The spinal strap thereby serves as a stop to preclude withdrawal of the threaded shafts from the sidebars.

The appliance is assembled with the patient lying on his back on a support. The bars and strap are placed in position to support the patient in the manner, and the tension of the strap adjusted as described above.

With the patient positioned over the spinal strap with shoulders and hips supported by the shoulder bar and hip bar, respectively, the table or bed can be lowered to afford full access to the patient's torso. The clearance and access thus afforded can allow for bed cleaning, the taking of various body readings and for various patient treatments.

### DETAILED DESCRIPTION OF THE DRAWINGS

The objects and features of the present invention will become more manifest to those skilled in the art upon a reading of the following descriptions taken n connection with the following drawings and wherein:

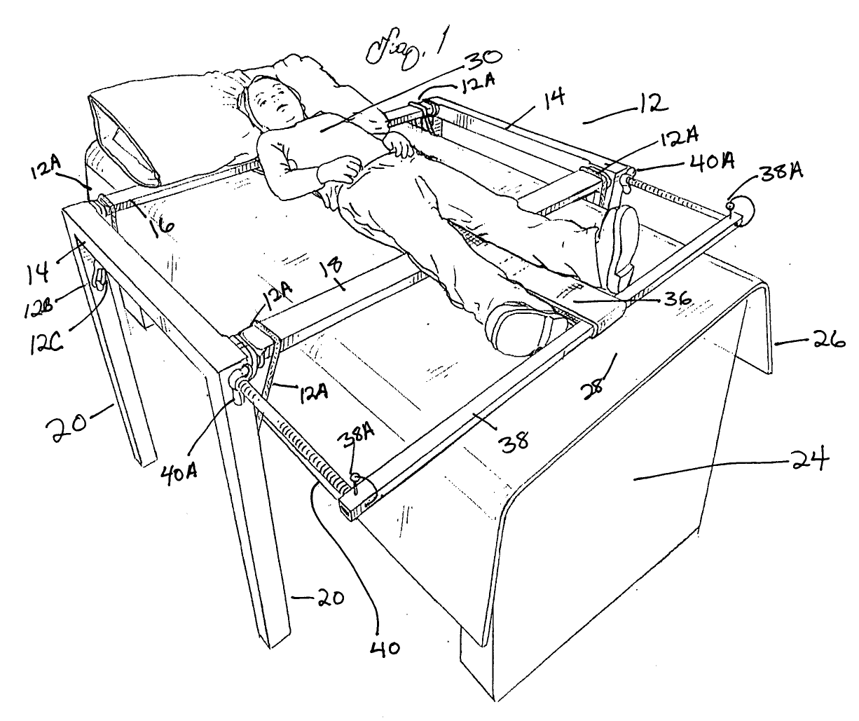

FIG. 1 is an isometric view of the appliance assembled around a surface supporting a patient;

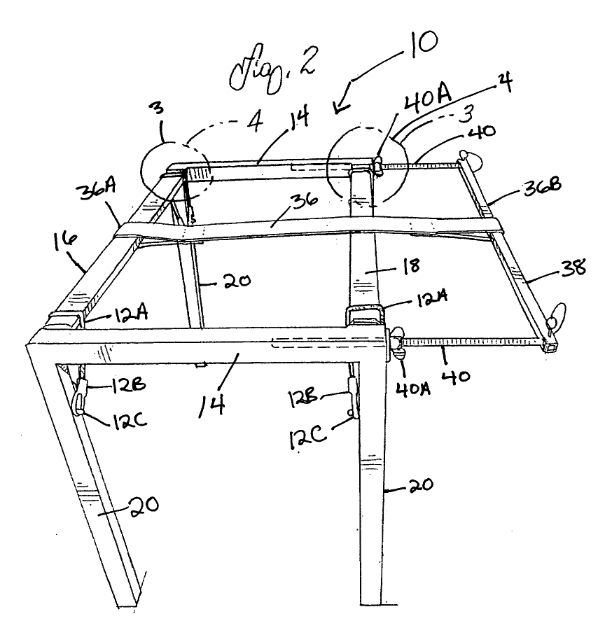

FIG. 2 is an isometric view of the appliance;

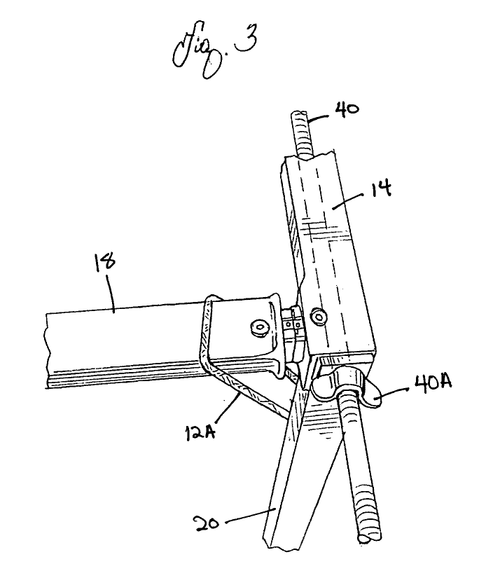

FIG. 3 is a detail of an assembled cross-bar, sidebar and leg which uses the first method of assembly;

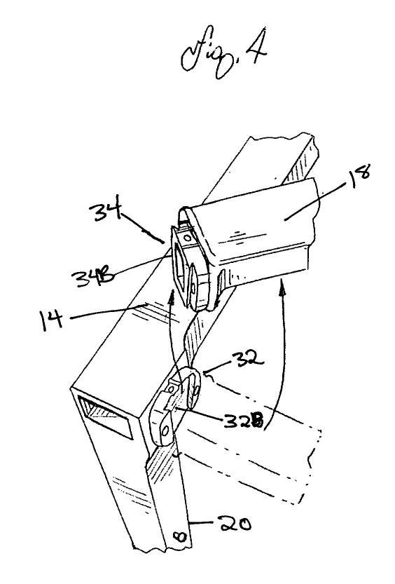

FIG. 4 is a detail of a cross-bar, sidebar and leg prior to assembly which utilize the first method of assembly;

FIG. 4 is an isometric end view of a cross-bar which utilize the second method of assembly;

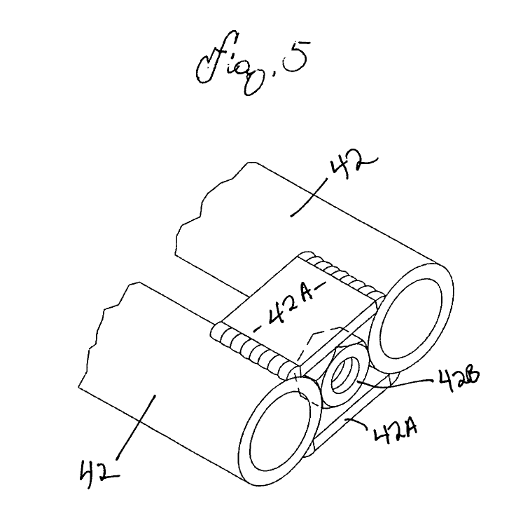

FIG. 5 is isometric end view of the ends of a cross bar and sidebar which utilize the second method of assembly;

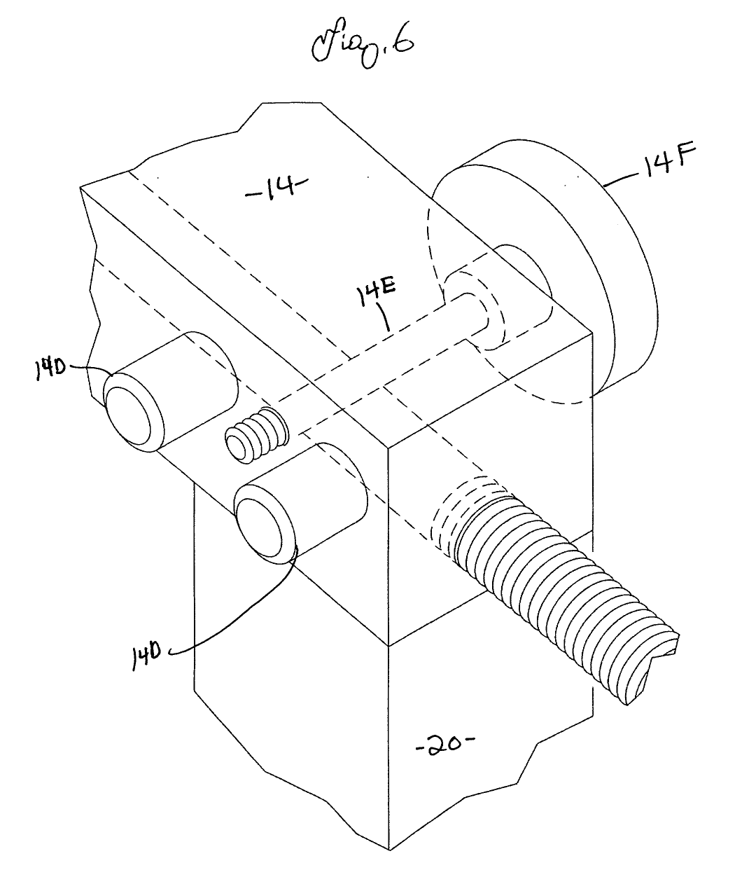

FIG. 6 is an isometric view end view of the ends of a side bar and leg which utilize the second method of assembly; and

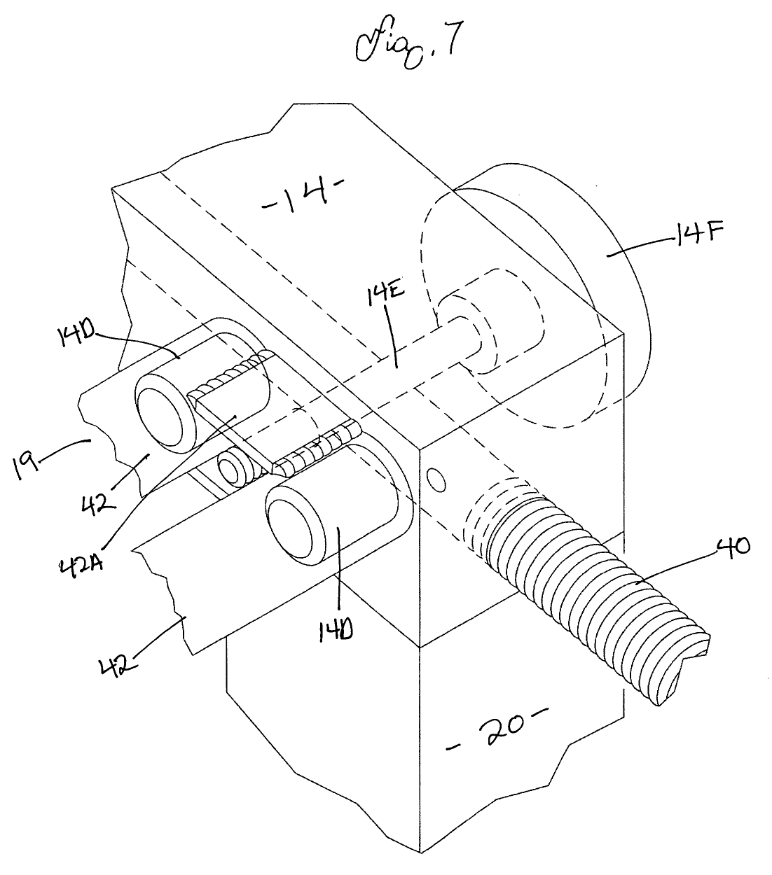

FIG. 7 is an isometric view end view of the ends of a side bar, cross-bar and leg which utilize the second method of assembly.

### DETAILED DESCRIPTION OF THE INVENTION

FIGS. 1-7 show apparatus 10 and its various features. Apparatus 10 has a frame 12 with two longitudinal sidebars 14 which form longitudinal sides of the frame. Cross-bars of a shoulder bar 16 and a hip bar 18 tie the two sidebars 14 together. A horizontal rectangular supporting surface 24 is shown with a covering 26 supports reclining patient 30. The length of sidebars 14, shoulder bar 16, and hip bar 18 are made such that frame 12 will closely enclose supporting surface 24. The length of legs 20 are equal and of a length such that support frame 12 will be essentially horizontal and be positioned immediately above supporting surface 28 when apparatus 10 is supported by an essentially horizontal floor. Legs 20 can either be made integral with other elements of frame 12, can be attached separately, and can be made adjustable in length whether made integral to the frame or not.

In FIG. 4 leg 20 is shown integral with sidebar 14. In FIGS. 1 and 2 legs 20 are integral with sidebar 14 on both sides of frame 12. The arrangement of leg 20 can be changed depending upon the type of support provided. In FIG. 3, leg 20 is shown secured within an end of sidebar 14.

FIG. 4 also shows one means of attaching one end of hip bar 18 to sidebar 14. This also applies to attaching the opposite end of hip bar 18 to the opposite sidebar 14, and to attaching both ends of shoulder bar to both sidebars. A female connector 32 is secured to the inner side of sidebar 14 by two bolts 32A through mating threaded holes in sidebar 14, not shown. Female connector part 32 has a generally trapezoid shaped opening 32B on the exposed side, is secured to sidebar 14 with the widest part of the trapezoid upward and has a upward extending lip from the lower edge. Male connector part 34 has a trapezoidal shaped extension 34B sized to fit within part 32B. Part 34 is secured to the end of hip bar 18 by two bolts 32A through mating threaded holes, not shown. Hip bar 18 is secured to sidebar 14 by dropping extension 34B into receptacle 32B from above where the weight of the sidebar will assist in holding it in position within the upward extending lip. The opposite end of hip bar 18 and both ends of shoulder bar 16 are attached to sidebars 14 using identical parts attached in the same manner.

As shown in FIGS. 1, 2 and 3, hip bar 18 can be further secured in place by an elastic bungee 12A which encircles the hip bar and is attached to leg 20 by a end fastener 12B, which is secured through hole by a bolt 12C. Both ends of shoulder bar 16 and hip bar 18 are secured to legs 20 by a bungee 12A in the same manner.

FIGS. 5, 6 and 7 show another and preferred means of attaching both ends of shoulder bars and hip bars between both sidebars 14. Shoulder bar 17 and hip bar 19 are substituted respectively for shoulder bar 16 and hip bar 18 in this second means, and are attached to the sidebars 14 using a different fastener permitted by their different construction. Shoulder bars 17 and hip bars 19 are both made from two tubes 42 which are the same length as shoulder bars 16 and hip bars 18. The two tubes 42 have a rectangular shaped connection 42A welded between the tubes at both ends on both sides to secure them together. A nut 42B is welded between tubes 42 and connection 42A at both ends of the bars and the nut is secured in place with the threaded opening aligned with the centerline of tubes 42. Extensions 14D from sidebar 14 mate with the interior of tubes 42. Threaded rod 14E mate with nut 42B on the interior end and has an attached knob 14F to rotate the rod for attaching and detaching the hip bar 19. The opposite end of hip bar 19 and both ends of shoulder bar 17 have similar arrangements as do the adjoining interior portions of sidebars 14 for attaching the parts together.

To provide for different height and different spacings between the shoulder and hips of patients, using the first or second connection means with either shoulder sidebar 14 and hip bar 16 or using shoulder sidebar 17 and hip bar 19, provides a plurality of locations for both the shoulder bars and the hip bars. This arrangement permits the bars to support the shoulder and the hip of any patient regardless of their height or their hip and shoulder spacing.

A flexible spinal strap 36 of flexible material extends longitudinally across the center of frame 12. Strap 36 has a loop 36A formed on one end and a loop 36B formed on the opposite end. Loop 36A encircles shoulder bar 16 and loop 36B encircles a tension bar 38. Tension bar 38 extends completely across frame 12 opposite sidebars 14 and is secured in place by threaded shafts 40 extending through the center of sidebars 14 through mating holes at the ends of the sidebars 14. The threaded shafts 40 engage mating threaded holes in tension bar 38, not shown, and are secured in place by bolts 38A threaded into mating holes.

Wing nuts 40A, engage shafts 40 having threads which mate with the threads on the shafts. Changing the position of the wing nuts 40A on shafts 40 provides a means for adjusting the position of tension bar 38, which is mounted between the ends of shafts 40, and change the amount of tension provided by strap 36.

In operation, patient 30 is lying on his back on the supporting surface 28, shoulder bar 16 with integral legs 10, and hip bar 18 or shoulder bar 17 and hip bar 10 are connected to sidebars 14. The parts are assembled with shoulder bar 16 or 17, hip bar 18 or 19 and strap 36 beneath the patient, and with loop 36A of strap 36 engaging shoulder bar 16 or 17. Shafts 40 engaged by wing nuts 40A are threaded into the ends of sidebars 14. Tension bar 38 is extended through loop 36B of strap 36 and connected to the ends of threaded shafts 40. Wing nuts 40A are then rotated to obtain the desired tension. Supporting surface 28 can then be removed leaving patient 30 supported by appliance 10.

As described above, sidebars 14 can be integral with legs 20, however the legs can be separable from the sidebars so that the frame 12, when not in use, can be fully disassembled. In either case, sidebars 14 are connected by either shoulder bar 16 or 17 and hip bar 18 or 19 respectively using either the first or second means described above.

As can be seen, with patient 30 positioned over spinal strap 36 with shoulders and hips supported by either shoulder bar 16 and hip bar 18 or by shoulder bar 17 and hi bar 19, the supporting surface can be lowered to afford more full access to the patient's torso. The clearance and access thus afforded can allow for bed cleaning, the taking of measurements and/or molding of the torso of the patient.

The above are just a few examples of the modifications and changes that are possible, and would readily occur to one skilled in the art, therefore it is contemplated that the appended claims will cover any such modifications or embodiments as fall within the true scope of the invention.

What is claimed is:

1. Apparatus for providing access to a reclining patient for treatment and observation comprising:
a) a framework essentially defining a plane arranged to provide access around the periphery of a patient reclining upon an essentially horizontal and an essentially rectangular shaped support surface which has a major and a minor axis, with the length of the patient extending essentially parallel to the major axis of the surface;
b) at least two support bars attached across the frame essentially parallel to the minor axis of the support surface at predetermined locations;
c) a flexible support positioned essentially under the spine of a reclining patient extending completely across the frame essentially parallel to the major axis of the support surface; and
d) framework support means for supporting the framework in an essentially horizontal attitude at a preselected elevation above an essentially planar and horizontal floor.

2. Apparatus as in claim 1 wherein the framework comprises an essentially rectangular shaped frame having a pair of longitudinally extending sidebars of a length at least equal to the distance between the shoulders and hips of the patient, the framework further having a pair of cross-bars of a length slightly greater than the minor dimension of the support surface.

3. Apparatus as in claim 2 further comprising first attachment means for attaching the cross-bars essentially perpendicular to the sidebars at predetermined locations along the length of the sidebars utilizing opposed mating male and female connector parts.

4. Apparatus as in claim 2 further comprising second attachment means for attaching the cross-bars essentially perpendicular to the sidebars at predetermined locations along the length of the sidebars utilizing opposed extensions from the sidebars which fit within recesses within the cross-bars, and a threaded rod from the sidebar which engages a threaded portion of the sidebar having external means for manually rotating the rod.

5. Apparatus as in claim 2 wherein the flexible patient support means comprises a strap having means for changing the strap tension.

6. Apparatus as in claim 5 wherein said means for changing the strap tension comprises a tension bar attached to the ends of each cross-bar by threaded rods engaging a threaded end of the cross bar being arranged such that rotating the rods will move the location of the tension bar with respect to the cross-bars, the strap having an end loop enclosing the tension bar.

7. Apparatus as in claim 3 further comprising means for connecting a bungee around an end of a cross-bar, a leg and a side bar.

8. Apparatus as in claim 1 wherein said support means comprises legs which are detachable from the frame.
